(19) United States
(12) Patent Application Publication
(10) Pub. No.: US 2006/0234373 A1
(43) Pub. Date: Oct. 19, 2006

(54) Treatment for diabetes

(76) Inventors: Alex Rabinovitch, Alberta (CA); Wilma Lucia Suarez-Pinzon, Alberta (CA); Antonio Cruz, Ontario (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/515,772

(22) PCT Filed: May 27, 2003
(86) PCT No.: PCT/US03/16660

§ 371 Date: Dec. 7, 2005

Related U.S. Application Data
(60) Provisional application No. 60/382,921, filed on May 24, 2002.
(60) Provisional application No. 60/384,357, filed on May 30, 2002.

(51) Int. Cl. C12N 5/06; C12N 5/08

(52) U.S. Cl. 435/325; 435/366

(57) ABSTRACT

Proliferating pancreatic islet cells obtained by the method of isolating a population of cells that preferably includes predominantly islet precursor cells that express one or more marker associated with an islet precursor cell and providing the precursor cells with one or more a pancreatic differentiation agent so that a population of cells is obtained that has a high proportion of cells with phenotypic characteristics of functional pancreatic islet β-cells. Optionally, the precursor cells are pretreated by providing them with one or more cell expansion agent to increase the number of cells in the population prior to differentiation. The pancreatic differentiation agent composition comprises a gastrin/CCK receptor ligand, e.g., a gastrin, in an amount sufficient to effect differentiation of pancreatic islet precursor cells to mature insulin-secreting cells. The cell expansion agent composition comprises one or more epidermal growth factor (EGF) receptor ligand in an amount sufficient to stimulate proliferation of the precursor cells. The methods of treatment include transplanting either undifferentiated precursor cells and providing the pancreatic differentiation agent either alone or in combination with the cell expansion agent in situ, or transplanting the functional pancreatic islet β-cells into the patient. The pancreatic islet β-cells can be used for drug screening, and replenishing pancreatic function in the context of clinical treatment.

### INTRODUCTION

### 1. Field of Invention

This invention relates generally to the field of cell biology of pancreatic islet precursor cells and methods for obtaining mature islet cells. More specifically, this invention relates to directed differentiation of human stem cells or other islet precursor cells that express one or more marker associated with islet precursor cells to functional pancreatic β-cells by providing one or both of a gastrin receptor ligand and an EGF receptor ligand and methods for use of the cells in the treatment of pancreatic disease, including diabetes mellitus, in an individual in need thereof. The method is exemplified by (a) providing human islet cells in vitro with a gastrin receptor ligand to stimulate insulin production prior to transplantation of the cells which optionally are provided with an EGF receptor ligand to expand the number of cells and (b) treatment of diabetes in vivo in a mouse model system for diabetes using a combination of a transplant of human islet cells and in vivo treatment with one or both of a gastrin receptor ligand and an EGF receptor ligand to promote proliferation of and/or insulin production by the transplanted islet cells.

### 2. Background

Diabetes is one of the most common endocrine diseases across all age groups and populations. In addition to the clinical morbidity and mortality, the economic cost of diabetes is huge, exceeding $90 billion per year in the US alone, and the prevalence of diabetes is expected to increase more than two-fold by the year 2010.

There are two major forms of diabetes mellitus: insulin-dependent (Type 1) diabetes mellitus (IDDM) which accounts for 5 to 10% of all cases, and non-insulin-dependent (Type 2) diabetes mellitus (NIDDM) which comprises roughly 90% of cases. Type 2 diabetes is associated with increasing age however there is a trend of increasing numbers of young people diagnosed with NIDDM, so-called maturity onset diabetes of the young (MODY). In both Type 1 and Type 2 cases, there is a loss of insulin secretion, either through destruction of the β-cells in the pancreas or defective secretion or production of insulin. In NIDDM, patients typically begin therapy by following a regimen of an optimal diet, weight reduction and exercise. Drug therapy is initiated when these measures no longer provide adequate metabolic control. Initial drug therapy includes sulfonylureas that stimulate β-cell insulin secretion, but also can include biguanides, β-glucosidase inhibitors, thiazolidenediones and combination therapy. It is noteworthy, however, that the progressive nature of the disease mechanisms operating in Type 2 diabetes are difficult to control. Over 50% of all drug-treated diabetics demonstrate poor glycemic control within six years, irrespective of the drug administered. Insulin therapy is regarded by many as the last resort in the treatment of Type 2 diabetes, and there is patient resistance to the use of insulin. Diabetic complications include those affecting the small blood vessels in the retina, kidney, and nerves, (microvascular complications), and those affecting the large blood vessels supplying the heart, brain, and lower limbs (macrovascular complications). Diabetic microvascular complications are the leading cause of new blindness in people 20-74 years old, and account for 35% of all new cases of end-stage renal disease. Over 60% of diabetics are affected by neuropathy. Diabetes accounts for 50% of all non-traumatic amputations in the US, primarily as a result of diabetic macrovascular disease, and diabetics have a death rate from coronary artery disease that is 2.5 times that of non-diabetics. Hyperglycemia is believed to initiate and accelerate progression of diabetic microvascular complications. Use of the various current treatment regimens cannot adequately control hyperglycemia and therefore does not prevent or decrease progression of diabetic complications.

Pancreatic islets develop from endodermal stem cells that lie in the fetal ductular pancreatic endothelium, which also contains pluripotent stem cells that develop into the exocrine pancreas. Teitelman and Lee, Developmental Biology, 121:454-466 (1987); Pictet and Rutter, Development of the embryonic endocrine pancreas, in Endocrinology, Handbook of Physiology, ed. R. O. Greep and E. B. Astwood (1972), American Physiological Society: Washington, D.C., p. 25-66. Islet development proceeds through discrete developmental stages during fetal gestation which are punctuated by dramatic transitions. The initial period is a protodifferentiated state which is characterized by the commitment of the pluripotent stem cells to the islet cell lineage, as manifested by the expression of insulin and glucagon by the protodifferentiated cells. These protodifferentiated cells comprise a population of committed islet precursor cells which express only low levels of islet specific gene products and lack the cytodifferentiation of mature islet cells. Pictet and Rutter, supra. Around day 16 in mouse gestation, the protodifferentiated pancreas begins a phase of rapid growth and differentiation characterized by cytodifferentiation of islet cells and a several hundred fold increase in islet specific gene expression. Histologically, islet formation (neogenesis) becomes apparent as proliferating islets bud from the pancreatic ducts (nesidioblastosis). Just before birth the rate of islet growth slows, and islet neogenesis and nesidioblastosis becomes much less apparent. Concomitant with this, the islets attain a fully difterentiated state with maximal levels of insulin gene expression. Therefore, similar to many organs, the completion of cellular differentiation is associated with reduced regenerative potential; the differentiated adult pancreas does not have either the same regenerative potential or proliferative capacity as the developing pancreas.

Since differentiation of protodifferentiated precursors occurs during late fetal development of the pancreas, the factors regulating islet differentiation are likely to be expressed in the pancreas during this period. One of the genes expressed during islet development encodes the gastrointestinal peptide, gastrin. Although gastrin acts in the adult as a gastric hormone regulating acid secretion, the major site of gastrin expression in the fetus is the pancreatic islets. Brand and Fuller, J. Biol. Chem., 263:5341-5347 (1988). Expression of gastrin in the pancreatic islets is transient. It is confined to the period when protodifferentiated islet precursors form differentiated islets. Although the significance of pancreatic gastrin in islet development is unknown, some clinical observations suggest a rule for gastrin in this islet development as follows. For example, hypergastrinemia caused by gastrin-expressing islet cell tumors and atrophic gastritis is associated with nesidioblastosis similar to that seen in differentiating fetal islets. Sacchi, et al., Virchows Archiv B, 48:261-276 (1985); and Heitz et al., Diabetes, 26:632-642 (1977). Further, an abnormal persistence of pancreatic gastrin has been documented in a case of infantile nesidioblastosis. Hollande, et al., Gastroenterology, 71:251-262 (1976). However, in neither observation was a causal relationship established between the nesidioblastosis and gastrin stimulation.

It is therefore of interest to identify agents that stimulate islet cell proliferation and/or regeneration for use in the treatment of early IDDM and in the prevention of β-cell deficiency in NIDDM.

### Relevant Literature

Three growth factors are implicated in the development of the fetal pancreas, gastrin, transforming growth factor a (TGF-α) and epidermal growth factor (EGF) (Brand and Fuller, J. Biol. Chem. 263:5341-5347). Transgenic mice over expressing TGF-α or gastrin alone did not demonstrate active islet cell growth, however mice expressing both transgenes displayed significantly increased islet cell mass (Wang et al, (1993) J Clin Invest 92:1349-1356). Bouwens and Pipeleens 1998) Diabetoligia 41:629-633 report that there is a high proportion of budding β-cells in the normal adult human pancreas and 15% of all β-cells were found as single units. Single β-cell foci are not commonly seen in adult (unstimulated) rat pancreas; Wang et al ((1995) Diabetologia 38:1405-1411) report a frequency of approximately 1% of total β-cell number.

Insulin independence in a Type 1 diabetic patient after encapsulated islet transplantation is described in Soon-Shiong et al (1994) Lancet 343:950-51. Also see Sasaki et al (Jun. 15, 1998) Transplantation 65(11):1510-1512; Zhou et al (May 1998) Am J Physiol 274(5 Pt 1):C1356-1362; Soon-Shiong et al (June 1990) Postgrad Med 87(8):133-134; Kendall et al (June 1996) Diabetes Metab 22(3):157-163; Sandler et al (June 1997) Transplantation 63(12):1712-1718; Suzuki et al (January 1998) Cell Transplant 7(1):47-52; Soon-Shiong et al (June 1993) Proc Natl Acad Sci USA 90(12):5843-5847; Soon-Shiong et al (November 1992) Transplantation 54(5):769-774; Soon-Shiong et al (October 1992) ASAIO J 38(4):851-854; Benhamou et al (June 1998) Diabetes Metab 24(3):215-224; Christiansen et al (December 1994) J Clin Endocrinol Metab 79(6):1561-1569; Fraga et al (April 1998) Transplantation 65(8):1060-1066; Korsgren et al (1993) Ups J Med Sci 98(1):39-52; Newgard et al (July 1997) Diabetologiz 40 Suppl 2:S42-S47.

### SUMMARY OF THE INVENTION

Methods and compositions for treating diabetes mellitus or other diseases of the pancreas in a patient in need thereof are provided in which one or both of a gastrin receptor ligand and an EGF receptor ligand are provided to stimulate islet cell regeneration and/or neogenesis. The compositions include a population of proliferating pancreatic islet cells obtained by the method of isolating a population of cells and providing the precursor cells with one or more pancreatic differentiation agent so that a population of functional pancreatic islet β-cells is obtained. Optionally, the precursor cells also are provided with one or more cell expansion agent to increase the number of cells in the population, generally prior to treatment with a differentiation agent. Preferably the population of cells has been enriched to include a higher percentage of islet precursor cells that express one or more marker associated with an islet precursor cell and thus have a high proportion of cells with phenotypic characteristics of functional pancreatic islet β-cells, including morphological features of β-cells, expressing surface markers characteristic of β-cells, and having enzymatic and biosynthetic activity important for pancreatic function. The pancreatic differentiation agent composition comprises a gastrin/CCK receptor ligand, e.g., a gastrin, in an amount sufficient to effect differentiation of pancreatic islet precursor cells to mature insulin-secreting cells. The cell expansion agent composition comprises one or more epidermal growth factor (EGF) receptor ligand in an amount sufficient to stimulate proliferation of the precursor cells. Optionally, both of these agents can be used at one or both of the expansion and differentiation steps. The methods of treatment include transplanting either undifferentiated precursor cells into a host animal and providing the pancreatic differentiation agent either alone or in combination with the cell expansion agent in vivo, or transplanting the functional pancreatic islet β-cells a host animal following provision with either one or both receptor ligand ex vivo. This system provides a source of functioning pancreatic islet β-cells for a variety of applications, such as drug screening, and replenishing pancreatic function in the context of clinical treatment, particularly of diabetes.

### BRIEF DESCRIPTION OF THE DRAWINGS

FIG. 1 shows the effects of TGF-α and gastrin on glucose tolerance in streptozotocin induced diabetic Wistar rats treated with PBS (solid black diamonds) or a combination of TGF-α and gastrin i.p. daily for 10 days (solid purple squares).

FIG. 2 shows the effect of TGF-α and gastrin treatment on β-cell neogenesis in three groups of treated Zucker rats together with the corresponding PBS controls (n=6 per group) as described in Example 4. The light blue bar represents lean TFG+gastrin, the magenta bar represents ob TGF+gastrin, the yellow bar represents the ob PBS control, the dark blue bar represents pre TFG+gastrin and the purple bar represents the lean PBS control. TGF-α and gastrin significantly increased the relative proportion of single β-cell foci in all the groups studied as compared to PBS-treated control animals. Groups 4 and 5 are significantly different (p<0.0015) as are Groups 1 and 2 (p<0.0041).

FIG. 3 shows the effect of TGF-α and gastrin treatment on β-cell neogenesis in lean and obese Zucker rats. β-cell neogenesis is quantified by differential counting of total β-cells and newly generated single β-cell foci and is expressed as a percentage of total β-cells counted. The percentage of single β-cell foci in lean Zucker rats treated with the growth factor combination was 10.5±0.9 compared to 3.9±1.1 (p=0.004) in the corresponding PBS control (FIGS. 3A and 3B). In the obese Zucker rats, the percentage of single β-cell foci in the pretreatment group was 8.7±1.3 vs. 4.2±1.1 (p=0.0015) in the corresponding control group (FIGS. 3C and 3D). FIG. 3E is a 400× magnification of the ductal region of FIG. 3C (indicated by an arrow) and provides clear evidence of the budding of insulin-containing β-cells from the ductal epithelial cells characteristic of β-cell neogenesis.

FIG. 4 shows that treatment with G1 decreases fasting blood glucose levels in chronically diabetic insulin-dependent NOD mice and prevents death 14 days after cessation of insulin therapy.

FIG. 5 shows that treatment with EGF decreases fasting blood glucose levels in chronically diabetic insulin-dependent NOD mice and prevents death 14 days after cessation of insulin therapy.

FIG. 6 shows that treatment with either E1 or G1 prevents increases in fasting blood glucose levels in NOD mice with recent-onset diabetes.

FIG. 7 shows that treatment with either E1 or G1 increases pancreatic insulin content in NOD mice with recent-onset diabetes.

FIG. 8 shows the results of EGF/gastrin treatment in diabetic mice. FIG. 8A is a set of line graphs showing the results of a glucose tolerance test, the graphs showing on the ordinate blood glucose (left graph) or plasma human C-peptide (right graph) as a function of time (up to 120 min.) on the abscissa, in NOD-Scid mice implanted with human islets and treated with gastrin/EGF (EGF, 30 μg/kg, and gastrin, 1000 μg/kg, solid symbols), or in control mice receiving vehicle only (open symbols). The right graph shows that gastrin/EGF improves insulin secretory response of human tissue. FIG. 8B is a bar graph showing that the content of human C-peptide in plasma is greater in EGF/gastrin-treated than in vehicle-treated mice.

FIG. 9 is a bar graph showing the insulin content, in μg/graft, of human islets implanted in NOD-Scid mice administered EGF+Gastrin (light gray bar), or vehicle (white bar), or in pre-implantation islets (dark gray bar). The data show that gastrin/EGF increases insulin content of human islets implanted in treated NOD-Scid mice compared to that in untreated NOD-Scid mice.

FIG. 10 is a bar graph of the percent β-cells (left graph) and total number of β-cells (right graph) in human islets implanted in mice as in FIG. 2. The data show that gastrin/EGF stimulates β-cell neogenesis in human islets implanted in treated NOD-SCID mice.

FIG. 11 is a set of microphotographs of insulin-positive cells (darkly stained) in an intact islet graft in NOD-SCID mice, or in isolated islet graft cells. The data show that gastrin/EGF induces an increase in the content of insulin-positive β-cells of implanted human islets.

FIG. 12 relates PDX-1 expression and insulin expression in treated cells. FIG. 12A is a set of photomicrographs that shows PDX-1 staining human islet cells and colocalization of PDX-1 and insulin expression in each of gastrin/EGF- and vehicle-treated cells. FIG. 12B is a bar graph showing PDX-1 expression at 8 weeks following transplantation in human islets implanted in NOD-SCID mice, during which the mice were treated with gastrin/EGF or with vehicle.

FIG. 13 is a set of line graphs showing the results of a glucose tolerance test, with blood glucose content (left panel) or plasma human C-peptide (right panel) shown on the ordinate as a function of time (up to 120 min.) on the abscissa, in NOD-SCID mice implanted with human islets and treated with low-dose gastrin/EGF (EGF, 30 μg/kg, and gastrin, 30 μg/kg; square symbols) or with vehicle (round symbols). The data show that gastrin/EGF even at a low dose improves insulin secretory response of human tissue.

### DETAILED DESCRIPTION OF PREFERRED EMBODIMENTS

The invention provides methods and compositions for treating diabetes mellitus and other degenerative pancreatic disorders in a patient in need thereof by providing a gastrin/CCK receptor ligand such a as gastrin, and/or an EGF receptor ligand, such as a TGF-α or an EGF, or a combination of both in an amount sufficient to effect differentiation of pancreatic islet precursor cells to mature insulin-secreting cells. When the composition is administered systemically, generally it is provided by injection, preferably intravenously, in a physiologically acceptable carrier. When the composition is expressed in situ, pancreatic islet precursor cells are transformed either in ex vivo or in vivo with one or more nucleic acid expression constructs in an expression vector which provides for expression of the desired receptor ligand(s) in the pancreatic islet precursor cells. As an example, the expression construct includes a coding sequence for a CCK receptor ligand, such as preprogastrin peptide precursor coding sequence which, following expression, is processed to gastrin or a coding sequence for an EGF receptor ligand such as TGF-α, together with transcripuonal and translational regulatory regions which provide for expression in the pancreatic islet precursor cells. The transcriptional regulatory region can be constitutive or induced, for example by increasing intracellular glucose concentrations, such as a transcriptional regulatory region from an insulin gene. Transformation is carried out using any suitable expression vector, for example, an adenoviral expression vector. When the transformation is carried out ex vivo, the transformed cells are implanted in the diabetic patient, for example using a kidney capsule.

Alternatively, pancreatic islet cells are treated ex vivo with a sufficient amount of a gastrin/CCK receptor ligand and/or an EGF receptor ligand to increase the number of precursor pancreatic β cells in the islets prior to implantation into the diabetic patient. As required, following expansion ex vivo the population of precursor pancreatic β-cells is differentiated in culture prior to implantation by contacting them with at least a gastrin receptor ligand. Whether expansion and/or differentiation are performed pre or post transplantation, the cells optionally are enriched prior to treatment for those cells that carry one or more marker for an islet precursor cell, such as a stem cell or a ductal cell expressing CK 19.

The subject invention offers advantages over existing treatment regimens for diabetic patients. By providing a means to stimulate adult pancreatic cells to regenerate not only is the need for traditional drug therapy (Type 2) or insulin therapy (Type 1 and Type 2) reduced or even eliminated, but the maintenance of normal blood glucose levels also may reduce some of the more debilitating complications of diabetes. By using one or both of expansion and differentiation of islet tissue or other islet precursor cells prior to or after transplantation, particularly in conjunction with enrichment of the precursor cell population to include a larger proportion of islet precursor cells, provides a means to decrease the number of scarce islets needed for transplantation. Additionally, not only is the variability of the population of cells used for transplantation decreased by the methods of the subject invention, but there is an increase in the reproducibility of the functional properties of the transplanted cells. Another advantage of the subject invention is that immune rejection can be reduced by, for example, xenotransplantation of porcine islets.

As used herein, the term “gastrin/CCK receptor ligand” encompasses compounds that stimulate the gastrin/CCK receptor. Examples of such gastrin/CCK receptor ligands include various forms of gastrin such as gastrin 34 (big gastrin), gastrin 17 (little gastrin), and gastrin 8 (mini gastrin); various forms of cholecystokinin such as CCK 58, CCK 33, CCK 22, CCK 12 and CCK 8; and other gastrin/CCK receptor ligands that either alone or in combination with EGF receptor ligands induce differentiation of cells in mature pancreas to form insulin-secreting islet cells. Also contemplated are active analogs, fragments and other modifications of the above, including both peptide and non-peptide agonists or partial agonists of the gastrin/CCK receptor such as A71378 (Lin et al, Am. J. Physiol. 258 (4 Pt 1): G648, 1990) that either alone or in combination with EGF receptor ligands induce differentiation of cells in mature pancreas to form insulin-secreting islet cells. Of particular interest is a gastrin derivative having a leucine substituted at position 15 in place of methionine. See U.S. Ser. No. 10/044,048 published Jul. 25, 2002, which disclosure is incorporated herein by reference. Gastrin/CCK receptor ligands also include compounds that increase the secretion of endogenous gastrins, cholecystokinins or similarly active peptides from sites of tissue storage. Examples of these are peptides, such as EGF and analogs and fragments thereof, and non-peptide small molecules, such as omeprazole, which inhibit gastric acid secretion and/or increase the number of gastrin/CCK receptors and soy bean trypsin inhibitor which increases CCK stimulation.

As used herein, the term “EGF receptor ligand” encompasses compounds that stimulate the EGF receptor such that when gastrin/CCK receptors in the same or adjacent tissues or in the same individual also are stimulated, neogenesis of insulin-producing pancreatic islet cells is induced. Stimulation of gastrin/CCK receptors can be directly by providing a gastrin/CCK receptor ligand, or indirectly, for example by inhibition of stomach acid secretion in vivo by endogenous and/or exogenous factors. Examples of EGF receptor ligands include EGF1-53, and fragments and active analogs thereof, including EGF1-48, EGF1-52, EGF1-49. See, for example, U.S. Pat. No. 5,434,135. Other analogs of interest include EGF having an amino acid sequence of length X, X being an integer that is at least 48 and not more than 53, such sequence (i) being substantially homologous to a portion of the amino acid sequence of human EGF from position 1 to position X-1 of human EGF and (ii) having at position X an amino acid residue different from that found in human EGF. Of particular interest is an analog of human EGF, wherein X is 51 and in which the amino acid residue at position X is other than glutamic acid, for example a neutral amino acid, a hydrophobic amino acid, or a charged amino acid. When X is 51, substitutions of interest include asparagine, glutamine, alanine, and serine (see PCT/US02/233097 published May 15, 2003, which disclosure is incorporated herein by reference). Other examples of an EGF receptor ligand include TGF-α receptor ligands (1-50) and fragments and active analogs thereof, including 1-48, 1-47 and other EGF receptor ligands such as amphiregulin and pox virus growth factor as well as other EGF receptor ligands that demonstrate the same synergistic activity with gastrin/CCK receptor ligands. These include active analogs, fragments and modifications of the above. For further background, see Carpenter and Wahl, Chapter 4 in Peptide Growth Factors (Eds. Sporn and Roberts), Springer Verlag, (1990).

A principal aspect of the invention is a method for treating diabetes mellitus in an individual in need thereof by providing to the individual a composition including a gastrin/CCK receptor ligand and/or an EGF receptor ligand in an amount sufficient to effect differentiation of pancreatic islet precursor cells to mature insulin-secreting cells. The cells so differentiated are residual latent islet precursor cells in the pancreatic duct. One embodiment comprises administering, preferably systemically, a differentiation regenerative amount of a gastrin/CCK receptor ligand and an EGF receptor ligand, preferably an EGF such as a substituted EGF-51, either alone or in combination to the individual.

Treatment of diabetes also can be effected by transplantation of purified islets or pancreatic islet precursor cells into a patient in need thereof. The cells for transplantation generally are obtained from a donor pancreas or are stem cells, obtained for example from umbilical cords, embryos or established cultured stem cell lines. The cells may be implanted by a route such as direct injection into an organ, for example, the pancreas, the kidney or the liver. Alternatively, the cells are administered by intravenous administration, for example, the cells are administered to the portal vein or the hepatic vein, for example, by percutaneous transhepatic injection into the portal vein. The cells can be expanded and/or differentiated into functional islet cells either post-implantation by providing the cells following transplantation or pre-implantation with a gastrin/CCK receptor ligand and/or an EGF receptor ligand.

If differentiated ex vivo the islet precursor cells, either stem cells or explanted pancreatic tissue can be partially or completely dissociated into isolated cells for either the differentiation step or the expansion step below before transplanting the pancreatic tissue so stimulated to a host mammal.

Prior to or concomitantly with contacting explanted pancreatic tissue with a differentiation-enhancing composition, the population of cells, particularly islet precursor cells in the explanted tissue, can expanded by providing a sufficient amount of an EGF receptor ligand with or without a gastrin/CCK receptor ligand, to induce mitogenesis. Optionally, the explanted pancreatic tissue can first be enriched in pancreatic islet precursor cells, particularly cells expressing a marker protein associated with islet precursor cells or ductal epithelial cells, for example CK19, nestin, CK7, CK8, CK18, carbonic anhydrase II, DU-PAN2, carbohydrate antigen 19-9 and mucin MUC1. Optionally, immortalized islet precursor cells can be prepared using methods known to those of skill in the art, for example by transformation with hTERT. Such cells can be can expanded with an EGF receptor ligand ex vivo and then stimulated with a gastrin receptor ligand to complete the differentiation process to fully mature islet cells in vivo or ex vivo prior to transplantation.

In another embodiment gastrin/CCK receptor ligand stimulation is effected by expression of a chimeric insulin promoter-gastrin fusion gene construct transgenically introduced into such precursor cells. In another embodiment EGF receptor ligand stimulation is effected by expression of an EGF receptor ligand gene transgenically introduced into the mammal. The sequence of the EGF gene is provided in U.S. Pat. No. 5,434,135.

In another embodiment stimulation by a gastrin/CCK receptor ligand and an EGF receptor ligand is effected by coexpression of (i) a preprogastrin peptide precursor gene and (ii) an EGF receptor ligand gene that have been stably introduced into the mammal.

In another aspect the invention relates to a method for effecting the differentiation of pancreatic islet precursor cells of a mammal by stimulating such cells with a combination of a gastrin/CCK receptor ligand and an EGF receptor ligand. In a preferred embodiment of this aspect, gastrin stimulation is effected by expression of a preprogastrin peptide precursor gene stably introduced into the mammal. The expression is under the control of the insulin promoter. EGF receptor ligand, e.g., TGF-α, stimulation is effected by expression of an EGF receptor ligand gene transgenically introduced into the mammal. In furtherance of the above, stimulation by a gastrin and a TGF-α is preferably affected by co-expression of (i) a preprogastrin peptide precursor gene and (ii) an EGF receptor ligand introduced into the mammal. Appropriate promoters capable of directing transcription of the genes include both viral promoters and cellular promoters. Viral promoters include the immediate early cytomegalovirus (CMV) promoter Boshart et al (1985) Cell 41:521-530), the SV40 promoter (Subramani et al (1981) Mol. Cell. Biol. 1:854-864) and the major late promoter from Adenovirus 2 (Kaufman and Sharp (1982) Mol. Cell. Biol. 2:1304-13199). Preferably, expression of one or both of the gastrin/CCK receptor ligand gene and the EGF receptor ligand gene is under the control of an insulin promoter.

Another aspect of the invention is a nucleic acid construct. This construct includes a nucleic acid sequence coding for a preprogastrin peptide precursor and an insulin transcriptional regulatory sequence, which is 5′ to and effective to support transcription of a sequence encoding the preprogastrin peptide precursor. Preferably, the insulin transcriptional regulatory sequence includes at least an insulin promoter. In a preferred embodiment the nucleic acid sequence coding for the preprogastrin peptide precursor comprises a polynucleotide sequence containing exons 2 and 3 of a human gastrin gene and optionally also including introns 1 and 2.

Another embodiment of the invention is an expression cassette comprising (i) a nucleic acid sequence coding for a mammalian EGF receptor ligand, e.g., TGF-α and a transcriptional regulatory sequence thereof; and (ii) a nucleic acid sequence coding for the preprogastrin peptide precursor and a transcriptional regulatory sequence thereof. Preferably, the transcriptional regulatory sequence for the EGF receptor ligand is a strong non-tissue specific promoter, such as a metallothionein promoter. Preferably, the transcriptional regulatory sequence for the preprogastrin peptide precursor is an insulin promoter. A preferred form of this embodiment is one wherein the nucleic acid sequence coding for the preprogastrin peptide precursor comprises a polynucleotide sequence containing introns 1 and 2 and exons 2 and 3 of the human gastrin gene.

Another aspect of the invention relates to a vector including the expression cassette comprising the preprogastrin peptide precursor coding sequence. This vector can be a plasmid such as pGem1 or can be a phage which has a transcriptional regulatory sequence including an insulin promoter.

Another aspect of this invention relates to a composition of vectors including (1) having the nucleic acid sequence coding for a mammalian EGF receptor ligand, e.g., TGF-α, under control of a strong non-tissue specific promoter, e.g., a metallothionein promoter; and a preprogastrin peptide precursor coding sequence under control of an insulin promoter. Each vector can be a plasmid, such as plasmid pGem1 or a phage in this aspect. Alternatively, the expression cassette or vector also can be inserted into a viral vector with the appropriate tissue trophism. Examples of viral vectors include adenovirus, Herpes simplex virus, adeno-associated virus, retrovirus, lentivirus, and the like. See Blomer et al (1996) Human Molecular Genetics 5 Spec. No: 1397-404; and Robbins et al (1998) Trends in Biotechnology 16:35-40. Adenovirus-mediated gene therapy has been used successfully to transiently correct the chloride transport defect in nasal epithelia of patients with cystic fibrosis. See Zabner eta. (1993) Cell 75:207-216.

Another aspect of the invention is a non-human mammal or mammalian tissue, including cells, thereof capable of expressing a stably integrated gene which encodes preprogastrin. Another embodiment of this aspect is a non-human mammal capable of coexpressing (i) a preprogastrin peptide precursor gene; and/or (ii) an EGF receptor ligand, e.g., a TGF-α gene that has been stably integrated into the non-human mammal, mammalian tissue or cells. The mammalian tissue or cells can be human tissue or cells.

### Therapeutic Administration and Compositions

Modes of administration include but are not limited to transdermal, intramuscular, intraperitoneal, intravenous, subcutaneous, intranasal, and oral routes. The compounds can be administered by any convenient route, for example by infusion or bolus injection by absorption through epithelial or mucocutaneous linings (e.g., oral mucosa, rectal and intestinal mucosa, etc.) and can be administered together with other biologically active agents. Administration is preferably systemic.

The present invention also provides pharmaceutical compositions. Such compositions comprise a therapeutically effective amount of a therapeutic, and a pharmaceutically acceptable carrier or excipient. Such a carrier includes but is not limited to saline, buffered saline, dextrose, water, glycerol, ethanol, and combinations thereof. The formulation should suit the mode of administration. Pharmaceutically acceptable carriers and formulations for use in the present invention are found in Remington's Pharnaceutical Sciences, Mack Publishing Company, Philadelphia, Pa., 17^{th} ed. (1985), which is incorporated herein by reference. For a brief review of methods for drug delivery, see Langer (1990) Science 249:1527-1533, which is incorporated herein by reference.

In preparing pharmaceutical compositions of the present invention, it may be desirable to modify the compositions of the present invention to alter their pharmacokinetics and biodistribution. For a general discussion of pharmacokinetics, see Remingtons's Pharmaceutical Sciences, supra, Chapters 37-39. A number of methods for altering pharmacokinetics and biodistribution are known to one of ordinary skill in the art (See, e.g., Langer, supra). Examples of such methods include protection of the agents in vesicles composed of substances such as proteins, lipids (for example, liposomes), carbohydrates, or synthetic polymers. For example, the agents of the present invention can be incorporated into liposomes in order to enhance their pharmacokinetics and biodistribution characteristics. A variety of methods are available for preparing liposomes, as described in, e.g., Szoka et al (1980) Am. Rev. Biophys. Bioeng. 9:467, U.S. Pat. Nos. 4,235,871, 4,501,728 and 4,837,028, all of which are incorporated herein by reference. Various other delivery systems are known and can be used to administer a therapeutic of the invention, e.g., microparticles, microcapsules and the like.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures such as a pharmaceutical composition adapted for intravenous administration to human beings. Typically, compositions for intravenous administration are solutions in sterile isotonic aqueous buffer. Where necessary, the composition also can include a solubilizing agent and a local anesthetic to ameliorate any pain at the site of the injection. Generally, the ingredients are supplied either separately or mixed together in unit dosage form, for example, as a dry lyophilized powder or water free concentrate in a hermetically sealed container such as an ampoule or sachette indicating the quality of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium and other divalent cations, isopropylamine, triethylamine, 2-ethylamino ethanol, histidine, procaine, etc.

The amount of the therapeutic of the invention which is effective in the treatment of a particular disorder or condition will depend on the nature of the disorder or condition, and can be determined by standard clinical techniques. The precise dose to be employed in the formulation also will depend on the route of administration, and the seriousness of the disease or disorder, and should be decided according to the judgment of the practitioner and each patient's circumstances. However, suitable dosage ranges for intravenous administration are generally about 0.01 to 500 micrograms of active compound per kilogram body weight for an EGF receptor ligand and generally about 0.1 to 5000 micrograms of active compound per kilogram body weight for a gastrin receptor ligand. Effective dosages can be extrapolated from dose-response curves derived from in vitro or animal model test systems. Suppositories generally contain active ingredient in the range of 0.5% to 10% weight; oral formulations preferably contain 10% to 95% active ingredient.

In the gene therapy methods of the invention, transfection in vivo is obtained by introducing a therapeutic transcription or expression vector into the mammalian host, either as naked DNA, complexed to lipid carriers, particularly cationic lipid carriers, or inserted into a viral vector, for example a recombinant adenovirus. The introduction into the mammalian host can be by any of several routes, including intravenous or intraperitoneal injection, intratracheally, intrathecally, parenterally, intraarticularly, intranasally, intramuscularly, topically, transdermally, application to any mucous membrane surface, corneal installation, etc. Of particular interest is the introduction of the therapeutic expression vector into a circulating bodily fluid or into a body orifice or cavity. Thus, intravenous administration and intrathecal administration are of particular interest since the vector may be widely disseminated following such routes of administration, and aerosol administration finds use with introduction into a body orifice or cavity. Particular cells and tissues can be targeted, depending upon the route of administration and the site of administration. For example, a tissue which is closest to the site of injection in the direction of blood flow can be transfected in the absence of any specific targeting. If lipid carriers are used, they can be modified to direct the complexes to particular types of cells using site-directing molecules. Thus, antibodies or ligands for particular receptors or other cell surface proteins may be employed, with a target cell associated with a particular surface protein.

Any physiologically acceptable medium may be employed for administering the DNA, recombinant viral vectors or lipid carriers, such as deionized water, saline, phosphate-buffered saline, 5% dextrose in water, and the like as described above for the pharmaceutical composition, depending upon the route of administration. Other components can be included in the formulation such as buffers, stabilizers, biocides, etc. These components have found extensive exemplification in the literature and need not be described in particular here. Any diluent or components of diluents that would cause aggregation of the complexes should be avoided, including high salt, chelating agents, and the like.

The amount of therapeutic vector used will be an amount sufficient to provide for a therapeutic level of expression in a target tissue. A therapeutic level of expression is a sufficient amount of expression to decrease blood glucose towards normal levels. In addition, the dose of the nucleic acid vector used must be sufficient to produce a desired level of transgene expression in the affected tissues in vivo. Other DNA sequences, such as adenovirus VA genes can be included in the administration medium and be co-transfected with the gene of interest. The presence of genes coding for the adenovirus VA gene product may significantly enhance the translation of mRNA transcribed from the expression cassette if this is desired.

A number of factors can affect the amount of expression in transfected tissue and thus can be used to modify the level of expression to fit a particular purpose. Where a high level of expression is desired, all factors can be optimized, where less expression is desired, one or more parameters can be altered so that the desired level of expression is attained. For example, if high expression would exceed the therapeutic window, then less than optimum conditions can be used.

The level and tissues of expression of the recombinant gene may be determined at the mRNA level as described above, and/or at the level of polypeptide or protein. Gene product may be quantitated by measuring its biological activity in tissues. For example, protein activity can be measured by inmnunoassay as described above, by biological assay such as blood glucose, or by identifying the gene product in transfected cells by immunostaining techniques such as probing with an antibody which specifically recognizes the gene product or a reporter gene product present in the expression cassette.

Typically, the therapeutic cassette is not integrated into the patient's genome. If necessary, the treatment can be repeated on an ad hoc basis depending upon the results achieved. If the treatment is repeated, the patient can be monitored to ensure that there is no adverse immune or other response to the treatment.

The invention also provides for methods for expanding a population of pancreatic β-cells in vitro. Upon isolation of the pancreas from a suitable donor, cells are isolated and grown in vitro. The cells which are employed are obtained from tissue samples from mammalian donors including human cadavers, porcine fetuses or another suitable source of pancreatic cells. If human cells are used, when possible the cells are major histocompatibility matched with the recipient. Purification of the cells can be accomplished by gradient separation after enzymatic (e.g., collagenase) digestion of the isolated pancreas. The purified cells are grown in media containing sufficient nutrients to allow for survival of the cells as well as a sufficient amount of a β-cell proliferation inducing composition containing a gastrin/CCK receptor ligand and EGF receptor ligand, to allow for formation of insulin secreting pancreatic β cells. According to the invention, following stimulation the insulin secreting pancreatic β cells can be directly expanded in culture prior to being transplanted into a patient in need thereof, or can be transplanted directly following treatment with β-cell proliferation inducing composition.

Methods of transplantation include transplanting insulin secreting pancreatic β-cells obtained into a patient in need thereof in combination with immunosuppressive agents, such as cyclosporine. The insulin producing cells also can be encapsulated in a semi-permeable membrane prior to transplantation. Such membranes permit insulin secretion from the encapsulated cells while protecting the cells from immune attack. The number of cells to be transplanted is estimated to be between 10,000 and 20,000 insulin producing β cells per kg of the patient. Repeated transplants may be required as necessary to maintain an effective therapeutic number of insulin secreting cells. The transplant recipient can also, according to the invention, be provided with a sufficient amount of a gastrin/CCK receptor ligand and an EGF receptor ligand to induce proliferation of the transplanted insulin secreting β cells.

The effect of treatment of diabetes can be evaluated as follows. Both the biological efficacy of the treatment modality as well as the clinical efficacy are evaluated, if possible. For example, disease manifests itself by increased blood sugar, the biological efficacy of the treatment therefore can be evaluated, for example, by observation of return of the evaluated blood glucose towards normal. The clinical efficacy, i.e. whether treatment of the underlying effect is effective in changing the course of disease, can be more difficult to measure. While the evaluation of the biological efficacy goes a long way as a surrogate endpoint for the clinical efficacy, it is not definitive. Thus, measuring a clinical endpoint which can give an indication of β-cell regeneration after, for example, a six-month period of time, can give an indication of the clinical efficacy of the treatment regimen.

The subject compositions can be provided as kits for use in one or more procedures. Kits for genetic therapy usually will include the therapeutic DNA construct either as naked DNA with or without mitochondrial targeting sequence peptides, as a recombinant viral vector or complexed to lipid carriers. Additionally, lipid carriers can be provided in separate containers for complexing with the provided DNA. The kits include a composition comprising an effective agent either as concentrates (including lyophilized compositions), which can be diluted further prior to use or they can be provided at the concentration of use, where the vials may include one or more dosages. Conveniently, in the kits single dosages can be provided in sterile vials so that the physician can employ the vials directly, where the vials will have the desired amount and concentration of agents. When the vials contain the formulation for direct use, usually there will be no need for other reagents for use with the method. Associated with such kits can be a notice in the form prescribed by a governmental agency regulating the manufacture, use or sale of pharmaceuticals or biological products, which notice reflects approval by the agency of manufacture, use or sale for human administration.

The following examples are offered by way of illustration and not by way of limitation.

### EXAMPLES

### Methods

The following methods were used in the examples set forth below except as otherwise noted.

### Animals

Normal Wistar and Zucker rats were allowed normal chow ad libidum with free access to water and were acclimatized for one week prior to initiation of each study. Freshly prepared streptozotocin at a dose of 80 mg/kg body weight was administered by I.V. five to seven days after induction of diabetes, the rats were randomly allocated into groups for subsequent treatment. In examples 1-4, TGF-α and rat gastrin were reconstituted in sterile normal saline containing 0.1% BSA. According to the predetermined treatment schedule for different studies, each animal received a single, daily i.p. injection of either TGF-α or gastrin alone (4.0 μg/kg body weight) or as a 1:1 (w/w) combination (total 8.0 μg/kg) or PBS for a period of 10 days.

Female NOD mice were fed under specific pathogen-free conditions and cared for properly in order to obtain 98% incidence of diabetes in the untreated female NOD mice. NOD diabetic mice were monitored for diabetes development by daily morning testing for glucosuria starting at 10 weeks of age by FBG. When glucosuria appears, the fasting blood glucose level (FBG) was measured and a FBG>6.6 mmol/l on two consecutive days was defined as diabetes. For the recent onset NOD model (Example 7), diabetic mice were typically selected for use at 14-18 weeks. For the chronic NOD model, diabetic mice were typically selected for use at 25 weeks of age (FBG levels are typically >30 mM) (Examples 5 and 6). Treatment for the female NOD-SCID (immunoincompetent sever combined immunodeficient) mice in Examples 8 and 9 were conducted when mice were 5-7 weeks of age. In Examples 8-9, mice were generally treated for 6-8 weeks, starting from immediately after transplantation, by administering each a dose of 30μg/kg of human mutant EGF having 51 amino acid residues, the residue at position 51 being an asparagine (described in application Ser. No. 10/000,840), and 30-1000 μg/kg of human gastrin analog hGastrin 1-17Leu 15, by intraperitoneal (i.p.) injection twice daily in saline/phosphate buffer.

### Blood Glucose

At the end of the treatment period, animals were subject to an overnight fast, and an intravenous (i.v.) or intraperitoneal (i.p.) glucose tolerance test was performed. Blood samples from fasting subjects were collected, as well as samples collected at different times after glucose injection. Samples were analyzed for blood glucose concentration and were then prepared for assay of human insulin C peptide levels by specific radioimmunoassay, the assay having negligible cross reactivity with C peptide from mouse if required.

### Tissue Insulin Analysis

At the end of each study, the animals were sacrificed and the pancreas and human islet graft (if implanted) removed and weighed.

Small biopsies were taken from separate representative sites throughout the pancreas tissue and immediately snap-frozen in liquid nitrogen for immunohistochemistry, protein, and insulin determinations. Snap-frozen pancreatic samples were rapidly thawed, disrupted ultrasonically in deionized water and aliquots taken for protein determination and the homogenate subjected to acid/ethanol extraction prior to insulin determination by RIA, and the total pancreatic islet content was calculated.

Human islet grafts were either frozen and extracted to assay insulin content by immunoassay, or were fixed in formalin for histological analysis. Human islet grafts harvested for analysis were extracted in acid ethanol to assay insulin content by immunoassay.

### Immunohistochemical Analysis

Human islet grafts were harvested and dissociated into cellular preparations that were immunostained with an antibody specific for each of insulin, glucagon, amylase and cytokeratins (CK) 7 and 19. Immunohistochemical techniques were performed as described in Suarez-Pinzon WL et al. (Diabetes 49: 1810 -1818, 2000). The percent of each of the different cell types and the insulin content in the grafted tissue and in the cell preparations were determined by counting stained and coded slides, each slide containing at least 12,000 cells per sample, and a count of each slide performed at least in triplicate, under a bright field microscope using a 100× immersion oil objective. At least 6,000 cells were counted per preparation, and the counts were repeated in a blind coded fashion twice. Counts were compared to the corresponding values in the graft-cell preparations prior to implantation.

### Human Islet Preparation and Implantation

Human islets were prepared as described previously from pancreas tissue of human donors, as follows. Islets are isolated from human pancreases, obtained with informed consent of relatives, from brain-dead organ donors. The human ethics committee of the hospital has approved tissue procurement and experimental protocols. Pancreas removal from donors and islet isolation procedures were performed according to Lakey JRT et al, (1999) Cell transplant 8:285-292, and Ricordi C. et al. (1988) Diabetes 37:413-420.

Human islets were transplanted into nondiabetic NOD/mice (2000 islet equiv) by implantation under the kidney capsule. Typically, one human donor pancreas was used to transplant about 10 to about 12 mice.

### EXAMPLE 1

### Effects of In Vivo Treatment with TGF-α and Gastrin on Pancreatic Insulin Content in Normal Rats

This experiment was designed to study the effects on pancreatic insulin content in non-diabetic animals treated with TGF-α, a gastrin, or a combination of TGF-α and a gastrin as compared to control animals (untreated). Groups (n=5) of normal Wistar rats were assigned to one of the following four treatment groups.
Group I: TGF-α: recombinant Human TGF-α was reconstituted in sterile saline containing 0.1% BSA and was administered i.p. at a dose of 0.8 μg/day for 10 days.
Group II: Gastrin: synthetic Rat Gastrin I was dissolved in very dilute ammonium hydroxide and reconstituted in sterile saline containing 0.1% BSA. It was administered i.p. at a dose of 0.8 μg/day for 10 days.
Group III: TGF-α+Gastrin: a combination of the above preparations was administered i.p. at the dose levels given above for 10 days.
Group IV: Control animals received an i.p. injection of vehicle alone for 10 days.

At the end of the study period (10 days), all animals were sacrificed and samples of pancreas taken as follows: five biopsy specimens (1-2 mg) of pancreatic tissue were taken from separate representative sites in each rat pancreas and immediately snap frozen in liquid nitrogen for analysis of insulin content. For analysis of pancreatic insulin content, the snap frozen pancreatic samples were rapidly thawed, disrupted ultrasonically in distilled water and aliquots taken for protein determination and acid/ethanol extraction prior to insulin radioimmunoassay (Green et al, (1983) Diabetes 32:685-690). Pancreatic insulin content values were corrected according to protein content and finally expressed as μg insulin/mg pancreatic protein. All values calculated as mean±SEM and statistical significance evaluated using Student's 2-sample t-test.

TABLE 1
Treatment of Normal Rats with TGF-α and Gastrin

| | Pancreatic Insulin Content |
|---|---|
| Treatment | (μg insulin/mg protein) |
| Control | 20.6 +/− 6.0 |
| TGF-α | 30.4 +/− 7.4* |
| Gastrin | 51.4 +/− 14.0** |
| TGF-α + Gastrin | 60.6 +/− 8.7*** |

*TGF-α vs. control, p = 0.34;
**gastrin vs. control, p = 0.11;
***combination of TGF-α and gastrin, p = 0.007.

As shown in Table 1, above, pancreatic insulin content was significantly increased (p=0.007) in the TGF-α+gastrin treated animals as compared to control animals; there was an approximately three-fold increase in pancreatic insulin content as compared to control animals. These data support the hypothesis that the combination of TFG-α and gastrin produces an increase in the functional islet β-cell volume. This increase reflects an overall condition of β-cell hyperplasia (increase in number) rather than β-cell hypertrophy (increase in size of individual β-cells).

### EXAMPLE 2

### Effect of In Vivo Treatment with a Combination of TGF-α and Gastrin on Pancreatic Insulin Content in Diabetic Animals

This experiment was designed to determine whether the combination of TGF-α and gastrin could increase pancreatic insulin content in diabetic animals (streptozotocin (STZ) treated) to levels comparable to those in normal (non-STZ treated) animals.

Normal Wistar rats received a single I.V. injection of STZ at a dose of 80 μg/Kg body weight. This dose of STZ was intended to ensure that the study animals were rendered diabetic but that they retained a functioning but reduced β-cell mass. The STZ was dissolved immediately before administration in ice-cold 10 mM citric acid buffer. The animals were monitored daily; persistent diabetes was indicated by glycosuria and confirmed by non-fasting blood glucose determinations. One week after induction of diabetes, rats were randomly allocated into two groups (n=6) as follows.
Group I: TGF-α+Gastrin: STZ diabetic rats were treated with a single i.p. injection of a combination of recombinant human TGF-α and synthetic rat Gastrin 1; both preparations were administered at a dose of 0.8 μg/day for 10 days.
Group II Control: STZ diabetic rats received an i.p. injection of vehicle alone for 10 days.

At the end of the study period, all animals were sacrificed and samples of pancreas taken and analyzed as described in Example 1; the results are given in Table 2.

TABLE 2
Treatment of Streptozotocin Rats with TGF-α and Gastrin

| | Pancreatic Insulin Content |
|---|---|
| Treatment | (μg Insulin/mg protein) |
| Control (STZ alone) | 6.06 ± 2.1 |
| STZ plus TGF-α + Gastrin | 26.7 ± 8.9 |

The induction of diabetes by STZ was successful and produced a moderate but sustained degree of hyperglycemia. Total insulinopaenia was not sought so as to ensure that the study animals retained a functioning, but reduced β-cell mass.

As shown in Table 2, above, the pancreatic insulin content of the control streptozotocin treated animals was less than one third that of normal rats (20.6±6.0 mg insulin/mg protein, see Table 1 above) as a result of destruction of β-cells by the STZ. In STZ animals treated with a combination of TGF-α and gastrin, the pancreatic insulin content was more than four-fold that of the animals which received STZ alone, and statistically the same as that of normal rats (see for example Table 1, above).

### EXAMPLE 3

### Effects of In Vivo Treatment with TGF-α and Gastrin on IPGTT in STZ-Induced Diabetic Animals

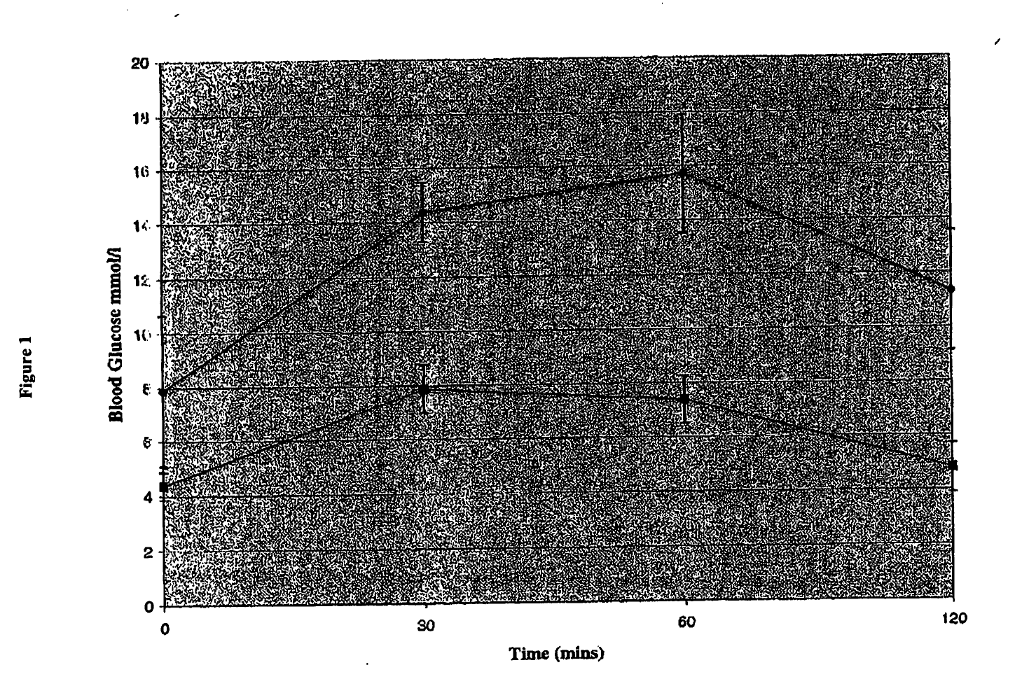

Two groups (average body weight 103 g) of STZ induced diabetic Wistar rats (n=6/group) were treated for 10 days with a daily i.p. injection of either a combination of TGF-α and gastrin or PBS. Fasting blood glucose was determined for all rats on days 0, 6, and 10. In order to establish that this insulin was both secreted and functional, IPGTT tests were performed. At day 10, intraperitoneal glucose tolerance tests (IPGTT) were performed following an overnight fast. Blood samples were obtained from the tail vein, before and 30, 60 and 120 minutes after administration of an i.p. glucose injection at a dose of 2 g/kg body weight. Blood glucose determinations were performed as above. The blood glucose levels were similar in both study groups at time 0 but the TFGα and gastrin treated rats demonstrated a 50% reduction in blood glucose values (FIG. 1), as compared to control rats at 30, 60, and 120 min. following the i.p. glucose load.

### EXAMPLE 4

### Effects of TGF-α and Gastrin on Body Weight Gain and Insulin Content in Diabetes Prone Animals

Zucker rats were obtained at 30 days of age approximately 10-15 days prior to development of obesity. Besides the diabetes prone Zucker rats (genotype fa/fa, autosomal recessive mutation for obesity and diabetes), lean non-diabetic littermates (genotype +/+) also were included in the study as described below. The rats were monitored daily for development of obesity and diabetes by determining body weight and blood glucose. The onset of diabetes in Zucker rats usually started between days 45-50 and was confirmed by a significant increase in blood glucose levels, as compared to the levels in age-matched lean controls.

The study included 5 groups of 5 rats each as described in Table 3. Groups 1 and 2 (lean, non-diabetic) were treated with a TGF-α and gastrin combination or PBS respectively from day 0 to day 10. Groups 3, 4 and 5 included obese, early diabetic Zucker rats, genotype fa/fa. Group 3 received a combination pretreatment for 15 days (day −15 to day 0) prior to onset of diabetes and continuing post onset of diabetes for 10 additional days (day 0 to day 10). Group 4 was treated with a combination of TGF-α and gastrin for 10 days after onset of diabetes and Group 5 was treated with PBS over the same time period. At the end of the study, the rats were sacrificed and the pancreas removed. Small biopsies were taken from separate representative sites for protein and insulin determinations as described above.

The body weight gain in obese diabetic Zucker rats with pretreatment, treatment only or with saline (groups 3, 4, and 5 in Table 3) did not show any significant differences among the groups. It is interesting to note that even prolonged treatment (25 days, group 3) with TGF-α+gastrin was without effect on normal weight gain. Within error limits body weight gain was identical in all the groups.

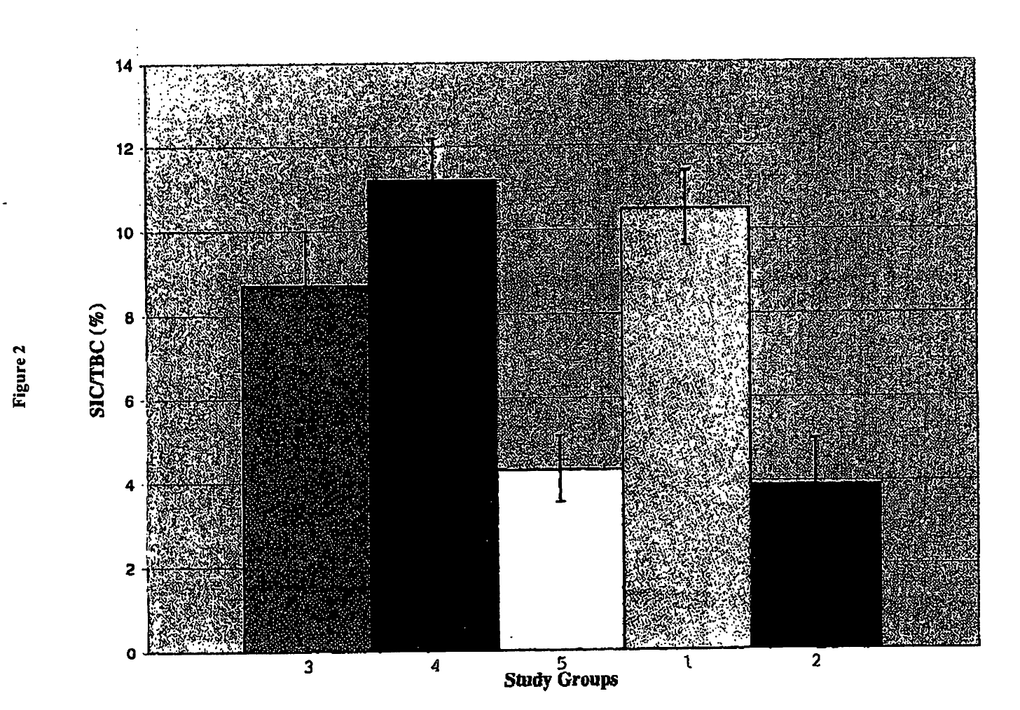

The effect of TGF-α+gastrin treatment on fasting blood glucose in the obese Zucker rats was compared to the corresponding PBS controls. Fasting blood glucose was first significantly increased by day 15 (4.0±0.6 vs. 5.0±0.2) and this time point was chosen as the starting time for the 10-day treatment period with TGF-α+gastrin or with PBS control. Fasting blood glucose levels were not significantly altered by the TGF-α+ gastrin treatment or by PBS. Fasting blood glucose values were lower in lean, as compared to obese animals whether or not they were treated with the growth factors or with PBS. These results are shown in Table 3, below, and FIG. 2.

TABLE 3

| | | | Pretreatment ± | | |
|---|---|---|---|---|---|
| | | | Treatment | PBS | Gain |
| Group | Geotype | Condition | (days) | Control | (% ± SE) |
| 1. | +/+ | lean, | None | Yes | 117 ± 2.1 |
| | | non-diabetic | | | |
| 2. | +/+ | lean, | 0 + 10 | No | 119 ± 1.9 |
| | | non-diabetic | | | |
| 3. | fa/fa | obese, | −15 + 10 | No | 202 ± 15 |
| | | early diabetic | | | |
| 4. | fa/fa | obese, | 0 + 10 | No | 119 ± 1.0 |
| | | early diabetic | | | |
| 5. | fa/fa | obese, | None | Yes | 129 ± 1.3 |
| | | early diabetic | | | |

### EXAMPLE 5

### Dose-Dependent Effects of In Vivo Treatment with Gastrin on Fasting Blood Glucose in NOD Mice with Chronic Insulin-Dependent Diabetes

The purpose of this experiment was to determine whether a gastrin alone can prevent development of severe hyperglycemia and death in NOD mice with chronic insulin-dependent diabetes. NOD mice with chronic insulin-dependent diabetes and maintained on insulin therapy were distributed into different treatment groups treated with: (i) vehicle (n=4); (ii) G1 1 μg/kg/day, given i.p. twice daily (n=4) for 28 days, (iii) G1 5 μg/kg/day, given i.p. twice daily (n=4) for 28 days, (iv) G1 10 μg/kg/day, given i.p. twice daily (n=4) for 28 days. Insulin therapy was stopped 14 days after commencement of treatment with G1. G1 is a 17 aa gastrin analog that is the same length as the native gastrin molecule but contains a single amino acid change at position 15 from met to leu.

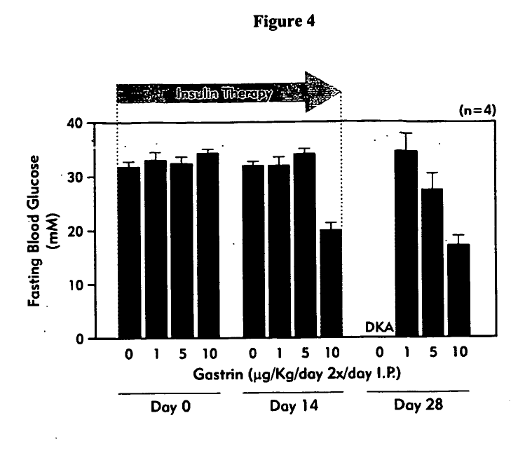

From day 0 to day 14, where the animals were maintained on insulin therapy, fasting blood glucose (FBG) levels for all treatment groups remained close to levels recorded at day 0 except for the group treated with 10 μg/kg/day of G1 which exhibited a decrease in FBG. At day 28, 14 days after the cessation of insulin therapy, all animals in the vehicle group died from diabetic ketoacidosis (DKA) since all these mice were completely dependent on insulin injections. However all mice treated with G1 survived without insulin treatment for 2 weeks. Fasting blood glucose levels for mice treated with 1 μg/kg/day of G1 remained elevated but there was a corresponding decrease of fasting blood glucose levels with increasing dose of G1 (5 and 10 μg/kg/day, respectively). See FIG. 4. These data show that treatment with gastrin significantly improves glucose control, without the use of insulin therapy, in chronically diabetic insulin-dependent NOD mice.

### EXAMPLE 6

### Dose-Dependent Effects of In Vivo Treatment with EGF on Fasting Blood Glucose in NOD Mice with Chronic Insulin-Dependent Diabetes

The purpose of this experiment is to determine whether an EGF can prevent development of severe hyperglycemia and death and can increase pancreatic insulin content in NOD mice with chronic insulin-dependent diabetes. NOD mice with chronic insulin-dependent diabetes and maintained on insulin therapy were distributed into different treatment groups treated with: (i) vehicle (n=4); (ii) E1 0.25 μg/kg/day, given i.p. twice daily (n=4) for 28 days, (iii) E1 1 μg/kg/day, given i.p. twice daily (n=4) for 28 days, (iv) E1 3 μg/kg/day, given i.p. twice daily (n=4) for 28 days. Insulin therapy was stopped 14 days after commencement of treatment with E1. E1 is a 51 amino acid EGF analog.

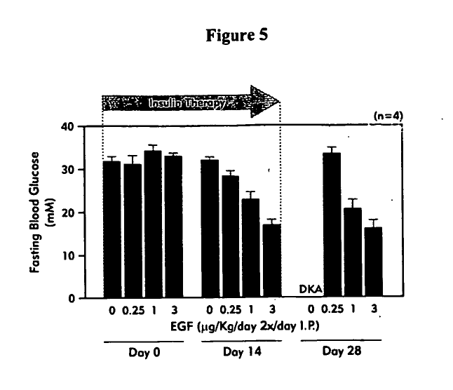

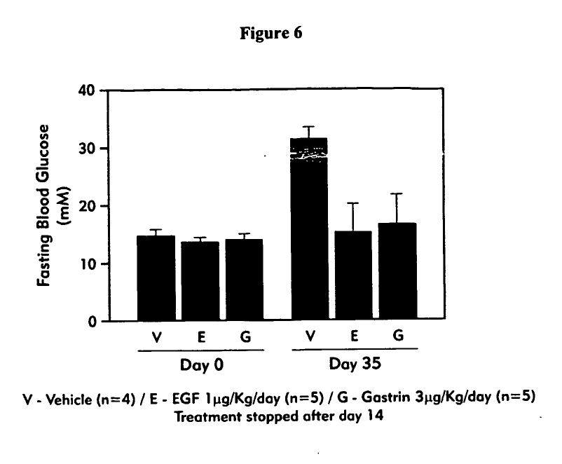

From day 0 to day 14, where the animals were maintained on insulin therapy, fasting blood glucose (FBG) levels for all groups treated with E1 demonstrated a dose-dependent decrease. At day 28, 14 days after the cessation of insulin therapy, all animals in the vehicle group died from diabetic ketoacidosis (DKA) since all these mice were completely dependent on insulin injections. In contrast, all NOD mice treated with E1 survived without insulin injection for 2 weeks. In addition, the decrease in FBG for the E1-treated groups remained steady at levels observed two weeks prior except for the group treated with 0.25 μg/kg/day of E1 in which the FBG remained elevated at day 28. See FIG. 5. These data show that treatment with EGF significantly improves glucose control, without the use of insulin therapy, in chronically diabetic insulin-dependent NOD mice.

### EXAMPLE 7

### Effects of an In Vivo Treatment with Gastrin or EGF on Fasting Blood Glucose and Pancreatic Insulin Content in NOD Mice with Recent Onset Diabetes

The purpose of this experiment was to determine whether either a gastrin or an EGF alone can improve diabetic conditions in NOD mice with recent onset diabetes. Non-obese diabetic (NOD) female mice were monitored for diabetes development (fasting blood glucose, FBG>6.6mmol/l). After diabetes onset, mice were treated with (i) vehicle (n=4), (ii) E1 1 μg/kg/day, given i.p. once daily (n=5) for 14 days, (iii) G1 1 μg/kg/day, given i.p. once daily (n=5) for 14 days. Mice did not receive insulin-replacement treatment. Fasting blood glucose levels and pancreatic insulin levels were monitored. E1 is a 51 amino acid EGF analog whereas G1 is a gastrin analog that is the same length as the native gastrin but contains a single amino acid change at position 15.

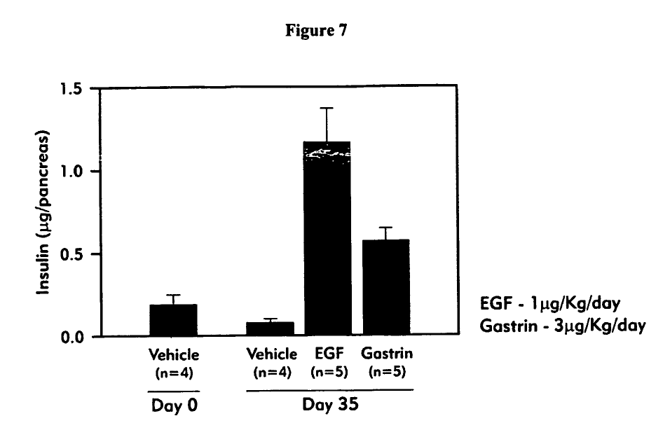

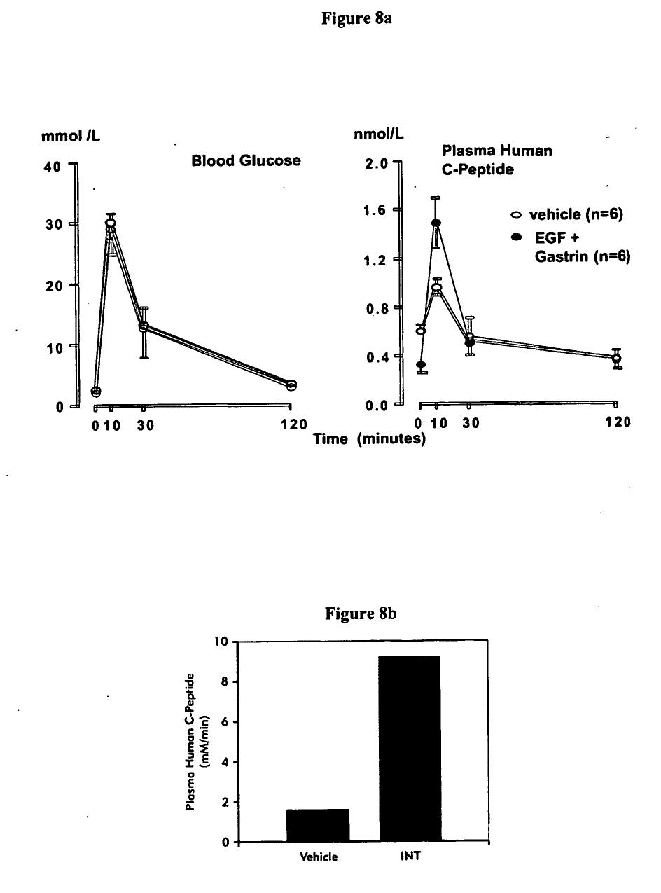

In the vehicle-treated control animals, fasting blood glucose (FBG) levels were doubled after 35 days. FBG levels of animals treated with either E1 or G1 remained close to values recorded at diabetes onset (day 0), in spite of ongoing destruction of islet cells in this animal model. Islet cell neogenesis stimulated by EGF or gastrin at the very least compensates the destruction of these cells. See FIG. 7. Pancreatic insulin levels also were measured in all animals. Pancreatic insulin levels for vehicle-treated controls decreased at day 35 due to destruction of β-cells, whereas animals treated with either E1 or G1 exhibited significantly elevated levels of pancreatic insulin levels in comparison to the pretreatment values. See FIG. 8. This study demonstrates that a short course (14 days) of treatment with either E1 or G1 after recent onset of diabetes in NOD mice can increase pancreatic insulin content and prevents progression of diabetic conditions for at least 3 weeks after therapy is stopped.

### EXAMPLE 8

### Characterization of Human Islet Grafts Transplanted to Mice and Treated In Vivo with a Gastrin and an EGF

Mice were transplanted with human islets (2000 islet equivalent) under the kidney capsule and were administered 1.5 μ/kg of glucose I.V. as a hyperglycemic stimulus. Blood samples were taken and were assayed as described above. The data show (FIG. 8A) that the blood glucose concentration time courses were similar in the EGF/gastrin mice and the vehicle treated mice. However, in response to the same hyperglycemic stimulus, the amount of human C-peptide released in plasma of EGF/gastrin-treated mice was more than five-fold greater than in plasma of vehicle-treated mice (9.2 and 1.8 nmoles/L/min, respectively; see FIGS. 8A and 8B), indicating that treatment was effective in stimulating insulin synthesis in the transplanted human islet. These data also show that the functional mass of transplanted human , cells was significantly greater in the gastrin/EGF treated mice as compared to the vehicle treated controls.

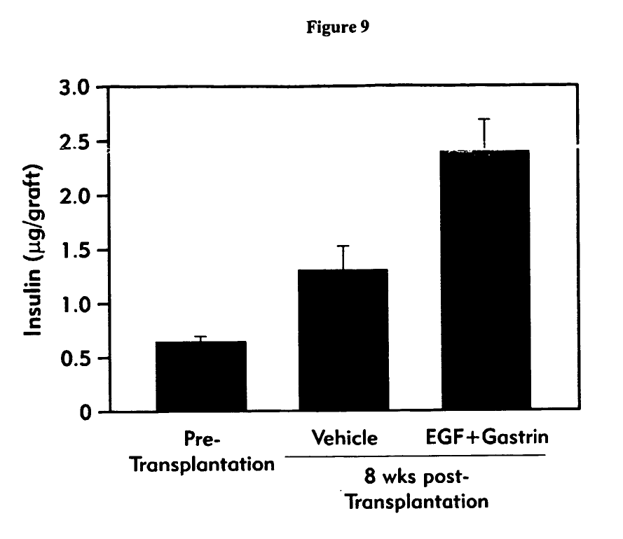

The insulin content of the human islet grafts was analyzed at 8 weeks post-implantation. Treatment with gastrin/EGF significantly increased the insulin content (2.42±0.28 μg per graft), as compared to the insulin content in islet grafts of mice treated with vehicle (1.34±0.21 μg per graft, p>0.02; FIG. 9) or to pre-implantation islets (less than 0.7 μg insulin per graft).

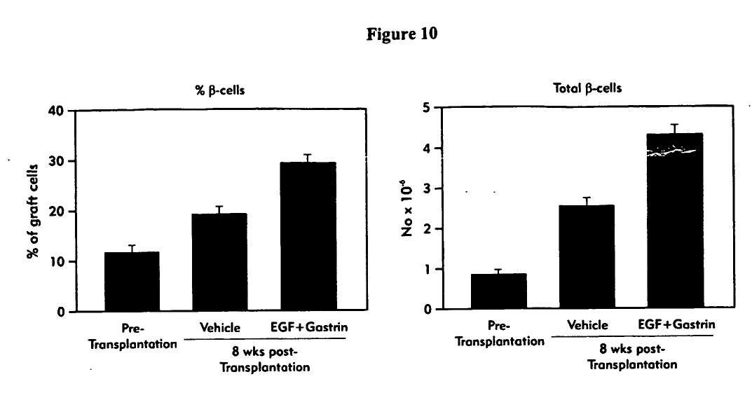

Immunocytochemical examination of the human islet grafts showed that gastrin/EGF treatment increased the percentage of β-cells observed in islet grafts (29.7±1.2%), as compared to the percentage of β cells in islet grafts in mice treated with vehicle (19.6±1.2%; FIG. 10). The total number of β cells observed in the grafts from EGF/gastrin treated mice (4.4±0.2×10⁶ β-cells) was also significantly greater than that observed in grafts from vehicle treated mice (2.6±0.2×10⁶ β-cells). Analysis of glucagon expressing α cells in the grafts from gastrin/EGF treated mice, as compared to the grafts from vehicle treated mice, revealed an increase in both the percent and the number of cells in response to gastrin/EGF treatment (Table 4). Further, the proportion of CK19 staining duct cells increased in the gastrin/EGF treated grafts. This is significant since CK 19/20 duct cells are thought to comprise a precursor population that gives rise to islet cells during islet neogenesis (Gmyr, V. et al., 2001, Diabetes 49:1671-80; Gmyr, V et al., Cell Transplantation, 2001, 10: 109-121). Pancreatic islets comprise a proportion of stem cells, variously estimated to be about one-fifth to one-third of the total cells in an islet. Table 4 also illustrates that upon treatment with a gastrin/EGF composition, the percentage of identified cells increases from about 53% or 59% to about 84%, due primarily to the increase in the percents of β and α secreting cells, indicating that gastrin/EGF treatment stimulates differentiation of stem cells in the islets into insulin secreting cells.

Compared to the preimplantation islets, by 8 weeks after transplantation there was a decrease in the number of acinar cells in both gastrin/EGF and vehicle treated mice. Overall, the cell composition in the gastrin/EGF grafts show a shift towards islet differentiation (62% islet and CK19 cells) compared to the vehicle treated (26%) and the pre-implantation tissue (20%).

TABLE 4
Cell Composition of Human Islet Transplant Grafts

| | % total cell count | | | | |
|---|---|---|---|---|---|
| | β | α | CK7 | CK19 | Amylase |
| Initial Transplant | 12 | 6 | 12 | 2 | 27 |
| 8 weeks Post Transplantation | | | | | |
| Vehicle | 20 | 4 | 15 | 2 | 12 |
| Gastrin/EGF | 30 | 18 | 8 | 16 | 12 |

| | Number of Cells (×10⁶) | | | |
|---|---|---|---|---|
| | | | Un- | % |
| | Total | Identified* | identified | Identified |
| Initial Transplant | 7.2 | 4.2 | 3.0 | 59 |
| 8 weeks Post Transplantation | | | | |
| Vehicle | 13.2 | 7.0 | 6.2 | 53 |
| Gastrin/EGF | 14.7 | 12.3 | 2.4 | 84 |

*Identified cells are β, α, CK7, CK19, and amylase cells.

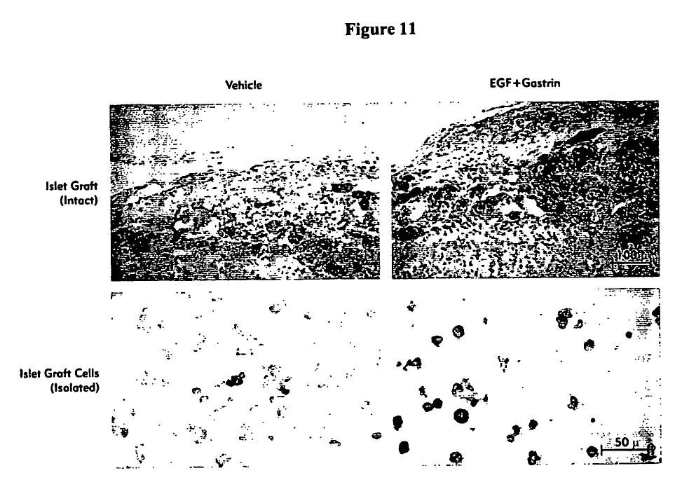

Treatment with gastrin/EGF induced increases in insulin-positive β-cells in human islets implanted in NOD-SCID mice. In FIG. 11, the upper panels (data from cells from an intact islet graft) show that insulin-staining β-cells were more abundant in a human islet graft from a mouse administered gastrin/EGF therapy, than from a mouse administered vehicle. The lower panels (data from isolated islet graft cells) show that the number of insulin-staining cells detected by innnunoperoxidase was much greater in a dissociated cell preparation from a human islet graft harvested from a mouse treated with gastrin/EGF as compared to a vehicle-treated mouse.

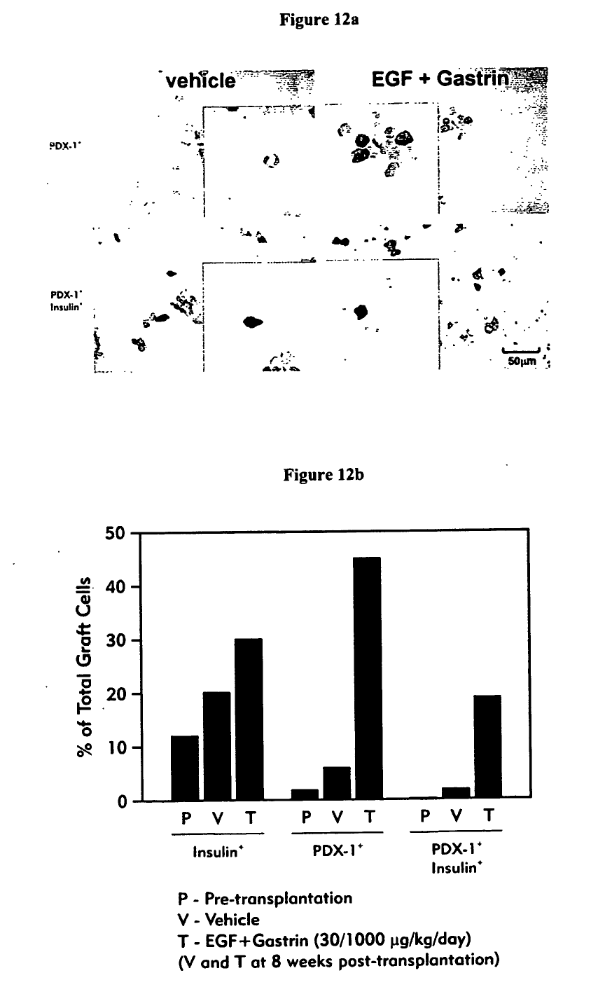

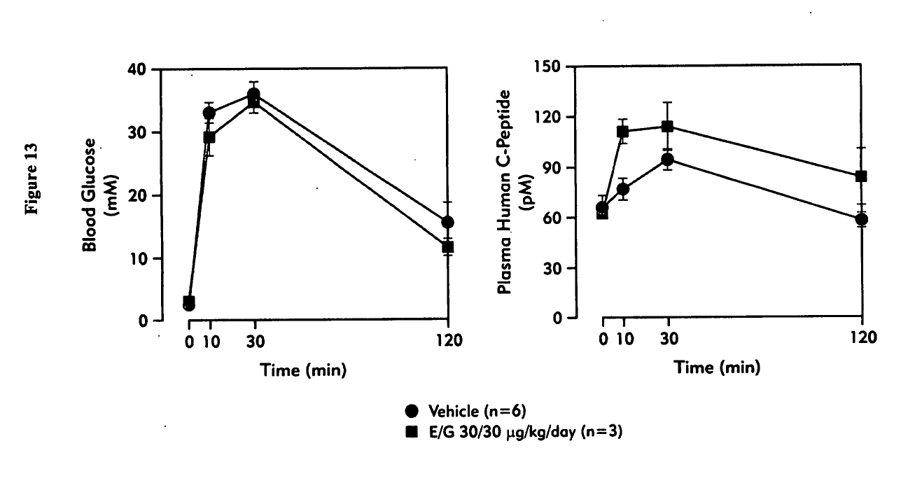

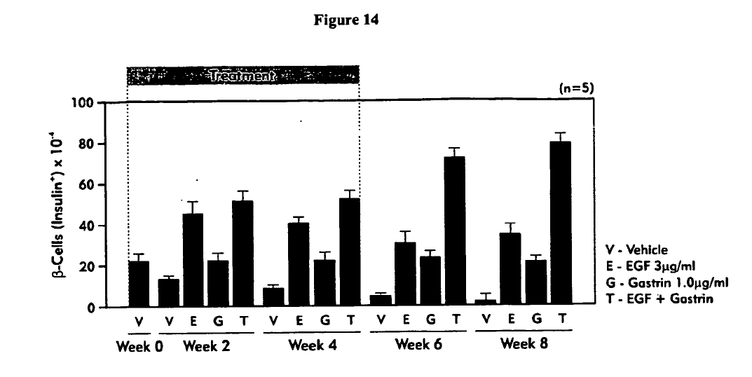

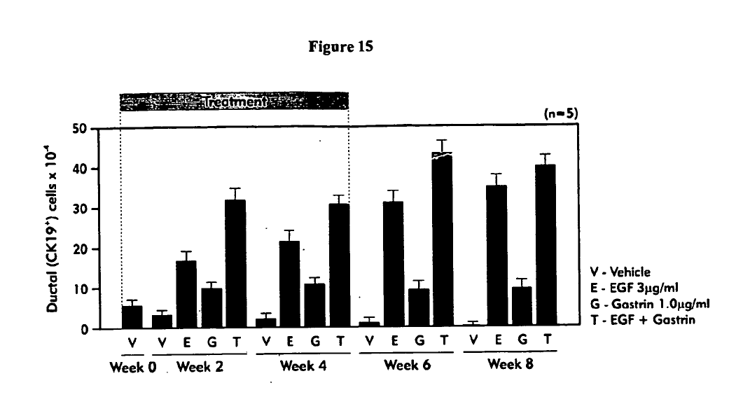

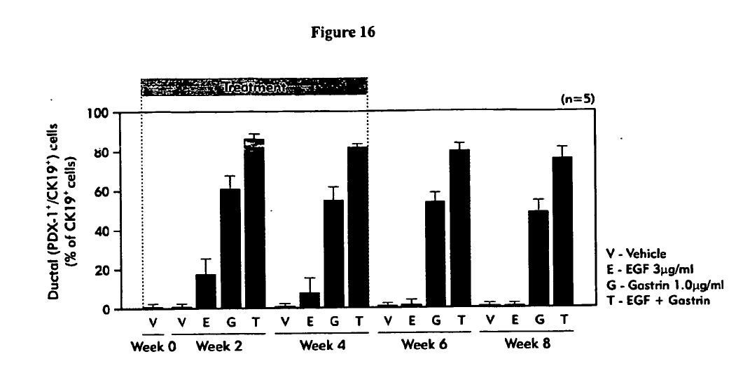

Treatment of mice with gastrin/EGF significantly increased expression of the amount of a marker for potential islet β-cells, the marker being precursor transcription factor PDX1 in human islet cells (FIGS. 12 and 13). This protein, encoded by the pancreatic and duodenal homeobox gene 1 (PDX-1), is central in regulating pancreatic development and islet cell function, and it regulates insulin gene expression.

Colocalization of PDX1 and insulin expression was also observed, as shown in both figures. These data demonstrate that gastrin/EGF induces PDX1 expression and increases β-cell mass in human islets implanted in NOD-SCID mice.

### EXAMPLE 9

### Administration of a Low Dose of Gastrin/EGF Stimulates Human β Cell Growth in Grafts of Human Tissue and Improves lnsulin-Secretory Response

Similar to the procedures Example 8 supra, NOD-SCID mice were treated for six weeks with either vehicle or with a low dose of gastrin/EGF (EGF, 30 μg/kg/day, and gastrin, 30 μg/kg/day, for 6 weeks given i.intraperitoneal in a single daily dose) and the insulin-secretory response measured.

After administration of 1.5 g/kg of IV glucose as a hyperglycemic stimulus, only a slight improvement in blood glucose tolerance was observed in mice treated with gastrin/EGF (FIG. 13). However, a significant improvement in the insulin-secretory response was observed, as evidenced by the greater release of human C-peptide in plasma of gastrin/EGF-treated mice as compared to that in plasma of vehicle-treated mice. Thus, even at a lower dose of gastrin, gastrin/EGF treatment results in an improvement in the insulin-secretory response of the human islet grafts.

### EXAMPLE 10

### Implantation and Differentiation of Human Stem Cells into Insulin Secreting Cells

The purpose of this experiment is to determine whether stem cells, for example from established cells lines, umbilical chords, or embryos, can be used in lleu of pancreatic islet grafts for implantation into diabetic patients, and differentiation into insulin-secretory cells by treatment with gastrin/EGF.

Stem cells from cell lines, or from umbilical cords are obtained from a closely related neonatal individual (child, cousin, niece or nephew) and implanted into each of a number of Type 1 diabetic patients. In a first iteration of this example, stem cells are implanted under the kidney capsule as in Example 1. Other methods of implantation in later iterations include I.V. administration, for example, into a portal or hepatic vein.

Groups of recipients are formed, the patients in each group of recipients being administered a dose of stem cells equivalent to about the number of stem cells in about 5 islets (about 10⁷ cells), in about 50 islets (about 10⁸ cells), in about 100 islets (about 2×10⁸ cells), in about 500 islets (about 10⁹ cells), in about 1000 islets (about 2×10⁹ cells), or in about 2000 islets (about 4×10⁹ cells), using the stem cell content of an islet as 25% of the total cell number, or about 2×10⁶ stem cells per islet (see Table 4 for total approximate cell number per islet).

Each implant recipient group is further divided into a control group to whom only vehicle (saline/phosphate buffer) is administered, and a treatment group. All patients are given standard IRB hospital review board clinical trial consent forms, and consent to be part of a trial in which they may receive a placebo. The treatment group receives a standard human protocol for a dose of a gastrin/EGF composition, about 3 μg/kg of EGF51N, and about 100 μg/kg of hGastrin 1-17Leu 15, i.p., twice daily in vehicle. Insulin therapy is continued in all recipient groups for about one month, and then is provided in reduced quantity, for example, about 50% to about 80% of the usual dose, concomitant with multiple daily monitorings and recordings of blood insulin and glucose. Additional insulin is administered as necessary to any patient, to maintain normal blood glucose, and all insulin doses and blood concentrations of insulin and glucose are recorded.

An end point determination indicates that, in the gastrin/EGF treatment group, initial administration of a smaller number of stem cells can provide sufficient insulin, compared to the number of stem cells required in the control group.

### EXAMPLE 11

### Effects of EGF (E1) and Gastrin (G1) on β-Cell Population of Isolated Human Islets Maintained in Culture

The purpose of this experiment was to determine whether treatment with EGF and gastrin can increase the β-cell population of human islets in vitro and by what mechanism. Islet cell preparations were isolated from human donor pancreases (n=5) and prepared according to the approved protocol used for preparation of human islets for islet cell transplantation (Lakey JRT et al (1999) Cell transplant 8:285-292, and Ricordi C. et al. (1988) Diabetes 37:413-420). Islet cells were seeded at a concentration of 1×10⁶ cells per dish and cultured for 4 weeks in serum free-MEM medium alone or supplemented with EGF (0.3 μg/ml), gastrin (1.0 μg/ml), or EGF+gastrin in combination for 4 weeks and maintained in culture for an additional 4 weeks.

Prior to treatment, the cellular composition of these islets determined by immunohistochemical antibody staining was: 7±2% glucagon⁺ α-cells, 23±3% insulin₊ β-cells, 17±2% CK7⁺ ductal cells, 6±1% CK19⁺ ductal cells, 33±2% amylase⁺ acinar cells, and 11±1% vimentin⁺ mesenchymal cells (mean±SE, n=5 donor pancreases). After 4 weeks, β-cell mass was increased by EGF+gastrin (+128%, p<0.001), and by EGF (+77%, p<0.01), but not by gastrin (−1%) or medium without EGF or gastrin (−60%, p<0.01).

Alter a further 4 weeks of incubation without EGF or gastrin added, there was a continued increase of β-cell mass in EGF+gastrin-treated islets (+244%, p<0.001) (FIG. 14). The EGF+gastrin-treated islet preparations also had an increase in CK 19⁺ ductal cells (+580%, p<0.001) (FIG. 15), together with increased expression of the islet transcription factor, PDX-1, in the CK 19⁺ ductal cells (there was no PDX expression before culture and this increased to 82±5% PDX-1⁺ after only 2 weeks of culture with EGF+gastrin) (FIG. 16). EGF+gastrin also increased the percentage of α-cells in the islet cultures, whereas the percentage of CK7⁺ ductal cells and acinar cells was decreased.

The percentage of β-cells significantly increased after 4 weeks of treatment by co-stimulation with E1 and G1 as compared to vehicle-treated control cultures. A further significant increase in the number of insulin-positive β-cells (almost 4-fold as compared to the β-cell numbers at the beginning of the treatment was observed) 4 weeks after the withdrawal of both peptides. A significant increase was observed in cells treated with both factors as compared to cells that were treated with either E1 or G1 alone. In contrast, a decrease in β-cell population was observed for the vehicle-treated islets at week 8.

The results of this experiment demonstrate that a combination therapy with EGF and gastrin significantly increases β-cell population of human islets in vitro. Even after withdrawal of the peptides after 4 Weeks, the β-cell number progressively increased in the cells that had been treated with co-stimulation of EGF and gastrin, showing that they have a synergistic and prolonged effect on the β-cell population.

Further it was found that EGF mainly increases the CK19+ ductal cell population (precursor cells), whereas Gastrin was mainly responsible for induction of PDX-1 expression on CK19+ ductal cells in human islets (FIG. 16). The protein encoded by the pancreatic and duodenal homeobox gene 1 (PDX-1) is central in regulating pancreatic development and islet cell function. PDX-1 regulates insulin gene expression and is involved in islet cell-specific expression of various genes.

These findings are in agreement with our transgenic mouse data where EGF receptor ligand alone (TGF-α) stimulated ductular cells but gastrin presence was necessary to complete the process of islet neogenesis initiated by an EGF receptor ligand.

These data strongly suggest that E1 and G1 may be used to expand human islet cell preparations in vitro for further transplantation in diabetic patients.

Diabetes mellitus is a disease in which the underlying physiological defect is a deficiency of β-cells as a result either of destruction of the β-cells due to auto-immune processes or of exhaustion of the potential for the β-cells to divide due to chronic stimulation from high circulating levels of glucose. The latter eventually leads to a situation when the process of β-cell renewal and/or replacement is compromised to the extent that there is an overall loss of β-cells and a concomitant decrease in the insulin content of the pancreas. The above results demonstrate that a combination of TGF-α and gastrin can be used to treat diabetes by stimulating the production of mature β-cells to restore the insulin content of the pancreas to non-diabetic levels.

The studies reported above demonstrate that complete islet cell neogenesis is reactivated in vivo in mammals in the ductular epithelium of the adult pancreas by stimulation with a gastrin/CCK receptor ligand, such as gastrin, and/or an EGF receptor ligand, such as TGF-α. Studies are reported on the transgenic over-expression of TGF-α and gastrin in the pancreas which elucidate the role of pancreatic gastrin expression in islet development and indicate that TGF-α and gastrin each play a role in regulating islet development. Thus, regenerative differentiation of residual pluripotent pancreatic ductal cells into mature insulin-secreting cells is a viable method for the treatment of diabetes mellitus, by therapeutic administration of this combination of factors or compositions which provide for their in situ expression within the pancreas.

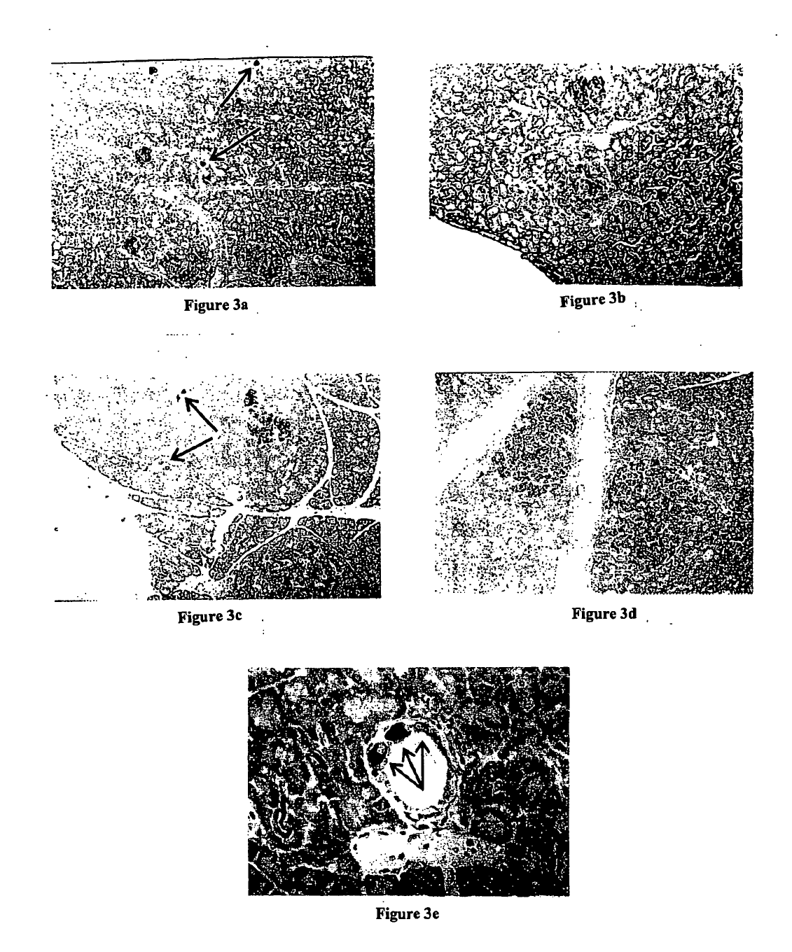

The results of treatment with TGF-α and gastrin in the Zucker rat model of Type 2 diabetes showed no significant differences in blood glucose levels between the treatment and control groups, probably reflecting the transient hypoglycemic effect following a prolonged period (18 hrs) of fasting. The immunohistochemical studies revealed significant increases in the number of single foci of insulin containing cells in the TGF-α and gastrin treated animals, as compared to control animals (FIG. 3). These findings demonstrated an increase in single β-cells in adult rat pancreas following treatment with TGF-α and gastrin. Interestingly, such single β-cell foci are not commonly seen in adult (unstimulated) rat pancreas. These findings support a therapeutic role for TGF-α and gastrin in Type 1 and Type 2 diabetes since treatment is targeted at both β-cell neogenesis and replication.

The present invention is not limited by the specific embodiments described herein. Modifications that become apparent from the foregoing description and accompanying figures fall within the scope of the claims.

Various publications are cited herein, the disclosures of which are incorporated by reference in their entirety.

What is claimed is:

1. A method for obtaining mammalian pancreatic cells comprising a plurality of functional mature β-cells, said method comprising:
providing a population of precursor mammalian pancreatic cells with at least one gastrin receptor ligand in an amount sufficient to effect differentiation of said precursor mammalian pancreatic cells, wherein said population of precursor mammalian pancreatic cells is enriched in cells that express at least one marker associated with precursor mammalian pancreatic cells, whereby a plurality of functional mature β-cells are obtained.

2. The method according to claim 2, wherein said marker is CK19.

3. The method according to claim 1, wherein said population of precursor mammalian pancreatic cells is enriched in cells that express at least one marker associated with precursor mammalian pancreatic cells by FACS.

4. The method according to claim 1, wherein said population of precursor mammalian pancreatic cells comprises a plurality of stem cells or ductal epithelial cells.

5. The method according to claim 4, wherein said stem cells comprise cells from one or more source selected from the group consisting of umbilical cords, embryos, and established stem cell lines.

6. The method according to claim 4, wherein one or more islets comprise said ductal epithelial cells.

7. The method according to claim 1, wherein said population of precursor mammalian pancreatic cells has been immortalized.

8. The method according to claim 1, wherein said population of precursor mammalian pancreatic cells is provided with at least one EGF receptor ligand in an amount sufficient to effect expansion of the population of said precursor mammalian pancreatic cells.

9. A method for obtaining a population of mammalian pancreatic cells comprising a plurality of functional mature β-cells, said method comprising:
providing a population of precursor mammalian pancreatic cells expressing at least one marker associated with precursor mammalian pancreatic cells with at least one gastrin receptor ligand in an amount sufficient to effect differentiation of said precursor mammalian pancreatic cells, wherein about 10% to about 20% of said cells express said marker, whereby a plurality of functional mature β-cells are obtained.

10. The method according to claim 9, wherein said cells are provided with at least one EGF receptor ligand in an amount sufficient to induce expansion of said population of functional mature β-cells by about 2-fold to about 5-fold.

11. The method according to claim 10, wherein expansion is about 3-fold to about 4-fold.

12. The method according to claim 9, wherein said providing is in vitro and said amount of said EGF receptor ligand is about 0.1 μg/ml to about 1.0 μg/ml and said amount of said gastrin receptor ligand is about 0.5 μg/ml to about 3 μg/ml.

13. The method according to claim 9, wherein said providing is in vitro and said amount of said EGF receptor ligand is about 0.2 μg/ml to about 0.5 μg/ml and said amount of said gastrin receptor ligand is about 0.6 μg/ml to about 1.5 μg/ml.

14. The method according to claim 9, wherein said plurality of functional mature β-cells express PDX-1.

15. The method according to claim 9, wherein precursor mammalian pancreatic cells are human or porcine.

16. The method according to claim 9, wherein the gastrin receptor ligand is human gastrin 1-17/Leu15.

17. The method according to claim 10, wherein the EGF receptor ligand is human EGF51N.

18. A composition comprising:
a cell culture comprising a plurality of proliferating mature pancreatic β cells, wherein said proliferating pancreatic β cells are obtained by the method of providing at least one gastrin receptor ligand and at least one EGF receptor ligand, and wherein said cell culture is enriched in CK19 ductal cells and have increased expression of PDX-1 as compared to cells not provided with a gastrin receptor ligand and an EGF receptor ligand.

19. A population of mammalian pancreatic precursor cells enriched to contain at least 20% precursor cell expressing CK19.

20. A method for screening for a compound that stimulates islet cell differentiation, said method comprising:
providing a population of mammalian pancreatic precursor cells with said compound and optionally at least one EGF receptor ligand; and
detecting expression of PDX-1 as an indication that said compound effects islet cell differentiation.
